(19) United States
(12) Patent Application Publication
(10) Pub. No.: US 2024/0350142 A1
(43) Pub. Date: Oct. 24, 2024

(54) A GUIDE CHANNEL FOR REGENERATIVE NERVE INTERFACE DEVICES

(71) Applicant: SCUOLA SUPERIORE DI STUDI UNIVERSITARI E DI PERFEZIONAMENTO SANT'ANNA, Pisa (IT)

(72) Inventors: Vincenzo LIONETTI, Pisa (IT); Silvestro MICERA, Firenze (IT); Eugenio REDOLFI RIVA, Pisa (IT); Ivo STRAUSS, Pisa (IT)

(73) Assignee: SCUOLA SUPERIORE DI STUDI UNIVERSITARI E DI PERFEZIONAMENTO SANT'ANNA, Pisa (IT)

(21) Appl. No.: 18/558,325

(22) PCT Filed: Apr. 27, 2022
(86) PCT No.: PCT/IB2022/053916

§ 371 Date: Oct. 31, 2023

(30) Foreign Application Priority Data
May 4, 2021 (IT) 102021000011297

(51) Int. Cl. A61B 17/11; A61B 17/00; A61L 31/00; A61L 31/04; A61L 31/10; A61L 31/16; A61N 1/05

(52) U.S. Cl. CPC A61B 17/1128; A61L 31/005; A61L 31/041; A61L 31/10; A61L 31/16; A61B 2017/00004; A61N 1/0556

(57) ABSTRACT

The present invention relates to a biocompatible and biodegradable tubular guide channel useful as a regenerative nerve interface to assist nerve regeneration and to support thin film electrodes capable of recording and stimulating electrical signals from the regenerated nerve. The invention further relates to a device containing the guide channel and thin film electrode, and to the process of manufacturing the guide channel and the device.

### FIELD OF THE INVENTION

The present invention relates generally to the field of biomedical devices, and more precisely relates to a guide channel for supporting a nerve to be regenerated in regenerative neural interface devices by surgical implantation in patients in need thereof, for example due to accidents, trauma, or neurodegenerative diseases. The invention also relates to devices and systems comprising such a guide channel, and a process for preparing them.

### BACKGROUND OF THE INVENTION

There are many causes that can lead to nerve damage, and this condition affects tens of millions of people in the world. Although the nerve tissue has been demonstrated to have a certain natural capacity for regeneration, in the event that the damaged nerve gap is longer than 10 mm, natural regeneration is not sufficient to recover function. In these cases, the damage and the consequent loss of function, e.g. loss of mobility of a limb, becomes permanent, unless surgical repairs are carried out, such as surgical grafting of another undamaged nerve or surgical transplantation of stem cells.

Such traditional surgeries usually have an unsatisfactory outcome in the face of the invasiveness of a surgical intervention, so recently different approaches have been proposed, which are potentially more effective from the point of view of repairing the damage but have yet to be optimised in clinical practice. One of these is based on the use of the so-called “Regenerative Neural interfaces” (RNIs): this term commonly refers to those devices that can record and/or stimulate a small population of axons, exploiting the regenerative properties of peripheral nerves. In case of nerve damage or nerve resection, the implantation of such devices aims to restore sensory-motor function or other nerve function lost, or otherwise impaired, such as the integration of the neural prosthesis with the amputated limb or the restoration of heart function following heart transplantation.

These devices are characterized by the presence of a channel that acts as a guidance for the nerve and induces its regeneration through an electrode mechanically supported by the channel itself. Unlike intrafascicular electrodes that require the insertion of an intact nerve, a regenerative neural device allows the nerve to be regenerated through the electrode, thus ensuring a considerable level of selectivity for stimulation/recording of electrical nerve signals [1].

The first devices of this type described in the literature contemplated the use of so-called sieve electrodes, called in this way because they consist of an array of holes with electrodes created all around the holes and placed transversely to a nerve guide channel made of silicone. By interfacing this device with a dissected nerve, the regrowth of axons within the holes was observed, allowing the stimulation of several nerve fascicles and the recording of their electrical activity. Since in these devices nerve regeneration is forced into small holes inside the electrode, the use of these sieve electrodes has remained confined to a few experiments [2]. In addition, long-term studies on this type of electrodes implanted in mice showed signs of axonopathy after about 6 months due to compression in some of the regenerated fibers [3].

Several other experimental studies have been proposed over time, but despite the success of these devices at the research level, long-term biocompatibility with tissues and stability of electrical performance over time remain critical aspects, which have so far prevented the translation of these devices into clinical practice. In order to achieve high stimulation/recording selectivity, the electrodes in these regenerative interfaces are characterized by an obstructive design, like the one of the sieve electrodes mentioned above, which is on the other hand necessary to force nerve regeneration through the active site. In addition, the materials used to date for both the nerve guide channel and the electrode have completely different chemical and physical properties compared to the surrounding biological tissue with which they should interact once implanted.

To address these limitations, different bio-hybrid approaches for the guide channels and different designs for the electrodes have been proposed in recent years [4-6]. For example, one of these approaches proposed a device with a thin film electrode made of highly transparent polyimide and placed longitudinally within a guide channel, so as to divide its interior into two separate corridors where two nerve fascicles can be regenerated independently of each other [7].

The design of the nerve guide channel, in particular, is crucial to ensure the biocompatibility of the regenerative interface with the tissues even in the long term. Moreover, this component should not only passively support the electrode during the nerve regeneration process, but should also be able to actively promote the regrowth of neurites. Therefore, biocompatible and biodegradable materials with chemical and physical properties that reproduce the native environment of neuronal cells have recently been proposed as suitable materials for guide channels [8]. Because of its biocompatibility, silicone is currently the gold standard as a material for realizing guide channels for regenerative neural interfaces, but its non-permeable nature prevents nerve regeneration over long gaps, e.g. >10 mm. In addition, the non-degradable nature of silicone exposes the patient to issues such as neuroma formation following compression of the regenerated nerve. Neuroma is a hyperplasia of Schwann cells and nerve fibers, which gives rise to a condition of severe pain, even with a minimal physical stimulation. Another major problem regarding the non-degradable nature of silicone, and more generally any non-resorbable implant, is the exposure to the risk of a second surgery to remove the implant due to the establishment of a chronic inflammatory response to the implanted device, and to the issues of micro-movements between the device and the surrounding tissue which, due to the chemical and physical differences between tissue and device, may damage the regenerated nerve and surrounding tissues over time.

To overcome such problems related to the use of silicone, the use of resorbable materials for the nerve guide channel, such as a decellularised layer of intestinal mucosa of the small intestinal wrapped around itself, combined with a hydrogel-based scaffold, was proposed in the US patent application published under No. US20180338765. These materials should support thin film electrodes by means of suture threads and thanks to the consistency of the hydrogel, whose function would be to avoid unwanted folding of the electrode. Although the inventors have reported on in vivo studies with these devices that still show good electrical properties after 6 months, difficulties due to the softness and poor handling of the guide channel may present themselves to the surgeon during the implantation step, especially in narrow anatomical districts, such as the cardiac region, which are characterized by the immediate proximity of vital organs and vessels, and therefore only allow limited, high-precision manoeuvres; or in body districts characterized by high cyclical mechanical stresses, such as regions close to the joints. In addition, guide channels of this type do not guarantee adequate mechanical support and the necessary stability to keep the electrode in the correct position, which condition might worsen the electrical performance of recording/stimulation of the nerve signal.

In the face of all the problems set out above for the known devices, their usefulness in nerve tissue regeneration is unquestionable and the potential applications are very promising. For these reasons, the need for new materials and a new design for the guide channel of regenerative nerve/neural interfaces is particularly felt in the sector in order to overcome the above-mentioned limitations.

### SUMMARY OF THE INVENTION

Now the inventors have developed a guide channel that manages to overcome the drawbacks highlighted above for the prior art thanks to the use of biodegradable and biocompatible materials, while providing a mechanically stable support for a thin film electrode and thus being able to create an effective, stable, biocompatible regenerative nerve interface device easier to surgically implant compared to the known devices.

An object of the invention is therefore represented by a tubular guide channel for supporting a nerve to be regenerated in regenerative nerve interface devices, the essential features of which are defined in the first of the appended claims. Further important features of the guide channel according to the invention are defined in the claims dependent on the first one.

A further object of the invention is a regenerative nerve interface device comprising the aforesaid guide channel and a thin film electrode, the essential features of which are defined in the respective independent claim appended thereto.

A further object of the invention is a process for manufacturing the aforesaid device, the essential features of which are defined by the respective independent claim appended thereto.

Still a further object of the invention is a system for supporting and regenerating damaged nerves comprising the aforesaid regenerative nerve interface device, means for controlling and processing the electrical signals coming from the device, means for the connection between the device and the control and processing means, the essential features of which are defined by the respective independent claim appended hereto.

Still a further object of the invention is a method for supporting and regenerating a damaged nerve comprising the steps of:
providing the aforesaid regenerative nerve interface device comprising a guide channel and a thin film electrode;
placing the device in a position adjacent to the damaged nerve;
suturing the guide channel of the aforesaid device, with the thin film electrode inside, around the damaged nerve; and
regenerating the damaged nerve and/or recording and/or stimulating the electrical activity of the nerve with the thin film electrode.

Other important features of the guide channel and the device, the manufacturing process thereof, the system and the method according to the invention are reported in the following detailed description, also with reference to the figures.

### BRIEF DESCRIPTION OF THE FIGURES

FIG. 1: shows the enlarged images of a portion of network mesh of poly (ε-caprolactone) (hereinafter PCL) printed at three different printhead speeds as described in Example 1 below (FIG. 1 a-c), and of the whole rectangular mesh obtained at one of the printing speeds (FIG. 1 d);

FIG. 2: shows the images of molds for realizing the guide channel of the invention, with different geometries and different wall thickness specifically to adapt them to experiments on pigs (FIG. 2 a,b) and on rats (FIG. 2 c,d); as described below, the geometry of the guide channel can be modulated more generally to make it suitable for different anatomical districts;

FIG. 3: shows the images of molds of different sizes with steel rod inserted inside to create the lumen of the guide channel (FIG. 3 a,b) and with the network mesh inserted in a special groove of one of the molds (FIG. 3 c);

FIG. 4: shows the images of two guide channels of the invention made in a cylindrical closed (FIG. 4 a) or open configuration with a longitudinal cut to form a cuff-like configuration (FIG. 4 b) to be closed with suture thread once the tubular element has been placed around the nerve to be regenerated;

FIG. 5: shows the images obtained by scanning electron microscope (SEM) in which the microstructure of the guide channel is visible, obtained at two different freeze-drying temperatures, at −20° C. (FIG. 5 a) and at −80° C. (FIG. 5 b); the scale bars are 1 mm and the arrows highlight the PCL network threads and the porous chitosan matrix;

FIG. 6: is a graph illustrating the kinetics of hydration of the guide channel by reporting the swelling index of the material over time by immersion in phosphate buffer solution (PBS) at 37° C., for the material of the invention and for a chitosan matrix without an internal network mesh as a comparison;

FIG. 7: shows the images of cutting tests of the guide channel according to various drawings and dimensions, in particular FIG. 7 a,b show guide channels for experiments on pigs rescaled with thinner thickness, and FIGS. 7 c,d show guide channels rescaled for rat experiments;

FIG. 8: shows in histogram form the compression resistance at different percent strain indices for the porous chitosan matrix without an internal network mesh as a reference, and for the same matrix with the internal network mesh of PCL prepared at the two different tested freeze-drying temperatures of −20° C. and −80° C.;

FIG. 9: shows in histogram form the average bending stiffness obtained in a 3-point bending test, also for the porous chitosan matrix without the internal network mesh as a reference, and for the same matrix with the internal network mesh of PCL prepared with 3D printer at two different speeds of the printhead;

FIG. 10: schematically illustrates a regenerative nerve interface system of the invention in a particular embodiment;

FIG. 11: schematically illustrates the steps of preparing an exemplary thin film electrode of an electrode of the invention, with photolithographic procedures described in detail in Example 3 below;

FIG. 12: schematically illustrates the steps of assembling the components of the regenerative nerve interface system of the invention.

### DETAILED DESCRIPTION OF THE INVENTION

### Definitions

In the context of the present invention, the term “biodegradable” means “capable of biological degradation, i.e. following the action of living organisms, to form harmless products”. With particular reference to the material of which the tubular guidance of the present invention is composed, and to the guidance itself, the term “biodegradable” means “capable of degradation once implanted in the human body, to form harmless, resorbable degradation products, after a period of time sufficient to perform the desired activity of regenerating a damaged nerve”.

The term “biocompatible” here means “capable of being compatible with a living system or a living tissue, without exerting on it any toxicity, damage or immunological reaction”. With particular reference to the material of which the tubular guidance of the present invention is composed, and to the guidance itself, the term “biocompatible” means “compatible with the surrounding tissues once implanted in the human body, without exerting damage, toxicity or immunological rejection”.

### DETAILED DESCRIPTION

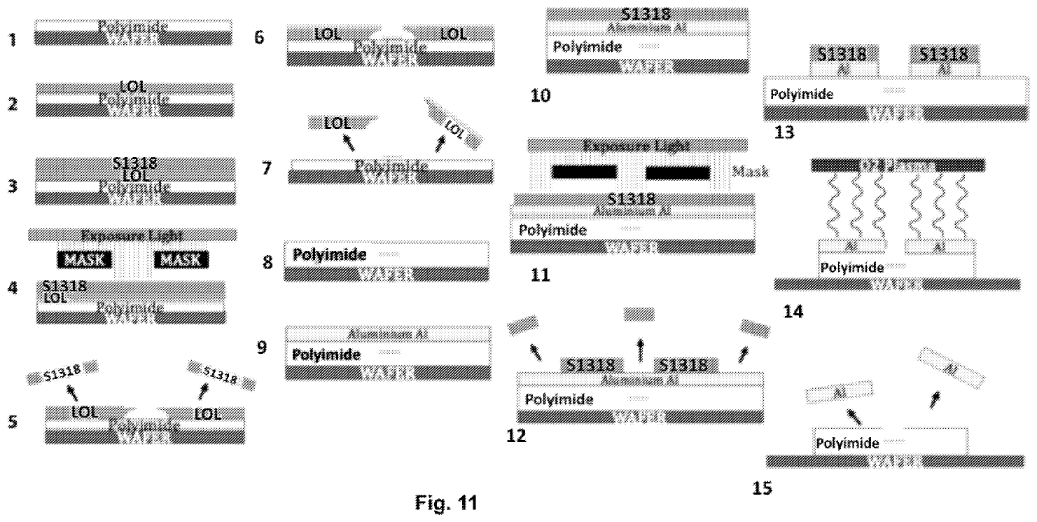

As mentioned above, this invention relates firstly to a guide channel adapted to support a nerve to be regenerated in regenerative nerve interface devices, illustrated for example in FIG. 4 and FIG. 10 appended hereto.

With reference to FIG. 10, which illustrates a particular embodiment of a regenerative nerve interface system comprising the guide channel 1 of this invention, the guide channel comprises a tubular element 11 comprising a natural hydrophilic polymer in the form of a porous matrix and a network mesh 12 comprising a synthetic thermoplastic polymer immersed in the tubular element 11; the guide channel 1 further comprises an internal lumen formed by the tubular element, with dimensions adapted to accommodate the nerve to be regenerated therein.

In one aspect of the invention, the network mesh of thermoplastic synthetic polymer also has a tubular shape and extends within the matrix over its entire surface. In another aspect of the invention, the network mesh extends within the matrix over a portion thereof.

The geometry of the network mesh of thermoplastic synthetic polymer of this invention may be any two-dimensional geometry, and preferably it is uniform throughout its extent as to thread thickness and geometry. In one particular aspect of the invention, the network mesh is uniformly designed as a honeycomb-like pattern.

Non-limiting examples of synthetic thermoplastic polymers of possible use according to the invention are selected from the group consisting of poly (:-caprolactone) (PCL), polylactic acid (PLA), polyglycolic acid (PGA), copolymers of lactic acid and polyglycolic acid (PLGA), and mixtures thereof. In a preferred aspect of the invention, the thermoplastic synthetic polymer is PCL.

Non-limiting examples of natural hydrophilic polymers of possible use according to the invention are selected from the group consisting of chitosan, collagen, hyaluronic acid, sodium alginate, dextran, cellulose, pectin, agarose, gellan, xanthan gum, silk, fibrin, keratin, chondroitin sulfate, and mixtures thereof. These polymers are widely used in tissue engineering and have proven to be perfectly biocompatible and resorbable, with a chemical and physical behaviour similar to the components of the extracellular matrix of biological tissues. In a preferred aspect of the invention, the natural hydrophilic polymer is chitosan. Chitosan, a derivative of chitin, is a natural polysaccharide that is also perfectly biocompatible and biodegradable and has also been demonstrated to have antibacterial properties. More generally, it has chemical and physical properties similar to natural glycosaminoglycans, and has been widely used for decades in tissue-engineering and drug release, so it is completely safe and suitable for the application now proposed by the inventors, as are the other natural polymers mentioned above.

In a particular embodiment of this invention, the tubular element 11 and/or the network mesh 12 may comprise one or more agents selected from, for example, anti-inflammatory agents, antibiotics, vitamins, antioxidants, agents capable of promoting nerve regeneration, such as growth factors, and stem cells, and mixtures thereof. Such agents may be incorporated, by methods known to any person of ordinary skill in the art, into the tubular element 11 and/or the network mesh 12, in the form of a powder, solution, dispersion or in the form of another pharmaceutical formulation which ensures a time-controlled gradual release. In one aspect of the invention, a formulation of one or more of the above-mentioned agents in the form of micro- or nano-particles may be incorporated into and immobilised in the porous matrix and/or on the surface of the network mesh.

The guide channel of the invention may have a tubular configuration with closed cylinder, with the cylinder bases open to allow the nerve to be regenerated to enter, or a cuff-like, longitudinally open cylinder configuration, as shown in FIG. 4b. This second configuration has advantages in clinical practice in that it can be implanted by having the nerve to be regenerated enter the lumen of the channel through the longitudinal cut, which can then be closed by suture thread. A further advantage of the latter configuration is that it facilitates visualisation of the nerve stumps to be bound to the lumen before the longitudinal closure of the guide channel with suture thread. More generally, in both configurations, the present guide channel is easier to implant than the known guide channels made of animal intestinal mucosa possibly reinforced with hydrogel; the synthetic polymer mesh network in fact gives the guide channel of this invention an ideal rigidity to facilitate the manipulation of the channel and the implantation thereof even in narrow anatomical areas, or in anatomical areas that are in close contact with vital organs like in the case of regeneration of the thoracic branch of the vagus nerve. The network mesh also offers the possibility of resting the stitches of the suture thread on the threads of the mesh rather than on the porous matrix of natural polymer, thus conferring better stability to the sutures which improves the overall mechanical stability of the device, also with respect to the electrode support, and minimizes the risks of rupture and tearing of the porous matrix. In the case of the cuff-like configuration of the guide channel, this avoids the risk of reopening once the longitudinal cut has been stitched, which would have a detrimental effect on nerve regeneration.

The invention also relates to a regenerative nerve interface device comprising the guide channel as described herein and a thin film electrode useful for recording the electrical activity of the nerve and stimulating its regeneration with electrical impulses that can be modulated on the basis of the recorded activity. Such a thin film electrode comprises an array of active contacts, a distal end and a proximal end and, in the present device, is inserted transversely within the tubular guide channel so that the 30 distal and proximal ends emerge outwards from opposite curved surfaces of the tubular channel while the array of active contacts is placed inside the channel, in a position such as to intercept the nerve to be regenerated.

In one embodiment of the invention illustrated in FIG. 10, the distal end 22 and/or the proximal end 23, on opposite sides of the active contacts 21 in the thin film electrode 2, are provided with one or more holes adapted to anchor the device to the surrounding tissues by means of suture thread.

Thin film electrodes adapted to realize this device for use in nerve regeneration can be selected by any person of ordinary skill in the art, and built ad hoc, e.g. using photolithographic procedures, such as the one exemplified below and illustrated in FIG. 11. In one aspect of the invention, the thin film electrode comprises a printed circuit board, covered with a polyimide film in which one or more conductive tracks forming an array of exposed active contacts of one or more conductive metal layers, for example made of gold, are etched. In one aspect of the invention, an additional coating may be added to the exposed active metal contacts, which further improves the electrical performance of the device at the electrode/tissue interface, such as for example reduction of impedance, increase of the amount of electric charge injected, improvement of the recording capacity of the nerve signal; further coatings of this type are selected from, for example, iridium oxide and conductive polymers, such as PEDOT: PSS, polypyrrole, polythiophene or polyaniline, and preferably PEDOT: PSS, and polypyrrole.

The present device is used to realize a regenerative nerve interface system, also forming part of this invention, and comprising the device described above, means for controlling and processing electrical signals coming from the device, comprising for example a printed circuit board, and means for the connection between the device and such control and processing means, such as conductive wires.

In one aspect of the invention between the thin film electrode and the control means the system further comprises at least one GND ground electrode.

In one aspect of the invention, at least the portion of the regenerative nerve interface system comprising control and connection means, for example conductive wires and printed circuit board is inserted within a thin sheath of a biocompatible material, optionally silicone. Preferably, said sheath is provided with one or more holes adapted to anchor the system to the surrounding tissues by means of suture thread to confer greater stability.

The present invention also covers the process for producing the regenerative nerve interface device described above, comprising the steps of:
i) providing a network mesh 12 of thermoplastic synthetic polymer by 3D printing at a predetermined printhead speed; and an aqueous solution of natural hydrophilic polymer;
ii) providing a mold adapted to form a cylindrical matrix of natural polymer with an internal lumen, optionally provided with a groove to hold the network mesh in place;
iii) inserting the rolled network mesh into said mold and pouring therein the solution of natural polymer;
iv) subjecting the mold to freeze-drying at a predetermined temperature, and extracting the guide channel 1 comprising a tubular element 11 comprising the natural polymer in the form of a porous matrix and a network mesh 12 of synthetic polymer immersed in the matrix;
v) bringing the guide channel to about 7.4 physiological pH by incubation in a basic aqueous solution;
vi) providing a thin film electrode 2 having an array of electrically active contacts 21, a distal end 22 and a proximal end 23; and
vii) inserting the electrode 2 transversely within the guide channel 1 so that the distal 22 and proximal 23 ends emerge from opposite curved surfaces of the guide channel outwards while the array of electrically active contacts 21 is placed within the guide channel.

In one aspect of this invention, the device manufacturing process also comprises a final step of fixing the electrode 2 to the guide channel 1, for example by gluing with a biocompatible glue, optionally followed by a step of hardening the glue. Advantageously, the gluing can be carried out at the point 6 of the guide channel, from which the distal end 22 of the electrode emerges outwards of the channel. In an alternative aspect of the invention, the fixing of the electrode 2 to the guide channel 1 can also be carried out by suturing with suture thread the electrode 2 to the network mesh using the holes provided on the electrode and the polymer filaments of the mesh.

In one aspect of the invention, the freeze-drying temperature in the above process is between about −20° C. and about −200° C. A freeze-drying temperature of about −20° C. is preferred to achieve higher compression resistance of the natural polymer matrix, and consequently better stability of the electrode within the guide channel. However, even lower freeze-drying temperatures, for example about −80° C. and up to about −200° C., which can be achieved for example with liquid nitrogen, can be advantageous for obtaining porous matrices in which the arrangement of the polymer chains is very directional and anisotropic, which are useful for example when it is wished to obtain matrices with adhered cells for tissue regeneration.

Optionally, the above-described process may comprise a porous matrix stabilization step, for example by cross-linking with appropriate cross-linking agents.

In another aspect of the invention, the speed of the 3D printer head for printing the PCL network mesh ranges from 0.4 mm/second to 1.0 mm/second. The inventors have experimentally verified that by varying the speed of the printhead, the weave of the network mesh, the thickness of the thread and consequently the bending stiffness of the guide channel are modulated. In particular, the lower the speed of the printhead, the thicker the mesh thread and the greater the bending stiffness of the guide channel. In another aspect of the invention, the geometry and the thickness of the network mesh threads in the present guide channel can also be modulated by varying the injection pressure of the molten polymeric material during the 3D printing step, for example in the region of about 6.4 bar, the injection pressure used in the experimental part below.

The system of this invention may be used to be surgically implanted in patients who have a severed or damaged nerve, or are otherwise in a condition where they May benefit from the present system for nerve regeneration. Thanks to the use of biodegradable and biocompatible materials for the realization of the guide channel, this nerve regenerative interface device and the system comprising it are perfectly biocompatible and resorbable by the body once their regenerative action has been completed. At the same time, the guide channel, thanks to the network mesh that coats it, has a stable structure and solidity that, in addition to facilitating surgical implantation operations, maintains the shape of the guide channel for the time necessary for nerve regeneration, without the structure collapsing or breaking, thereby creating an obstacle to the regeneration process. The inventors have been able to verify that this advantageous feature of this device is present both with the support element of the guide channel in a tubular closed configuration and in a tubular cuff-like open configuration.

Finally, the method for regenerating damaged nerves of the invention, based on the implantation of the present device, is extremely facilitated compared to the methods that can be achieved with known guide channels made of resorbable materials but which are too soft to be handled by the surgeon and placed correctly in position, while maintaining a stable support for the electrode over time. This advantageous aspect of the present invention takes on special significance in the case of deep anatomical districts which are difficult to reach, or in districts close to those of the joint ones, characterized by the presence of high cyclical mechanical stresses.

The following experimental part is given for illustrative but not limitative purposes of the present invention.

### Experimental Part

### Example 1

### Preparation of the Guide Channel of the Invention

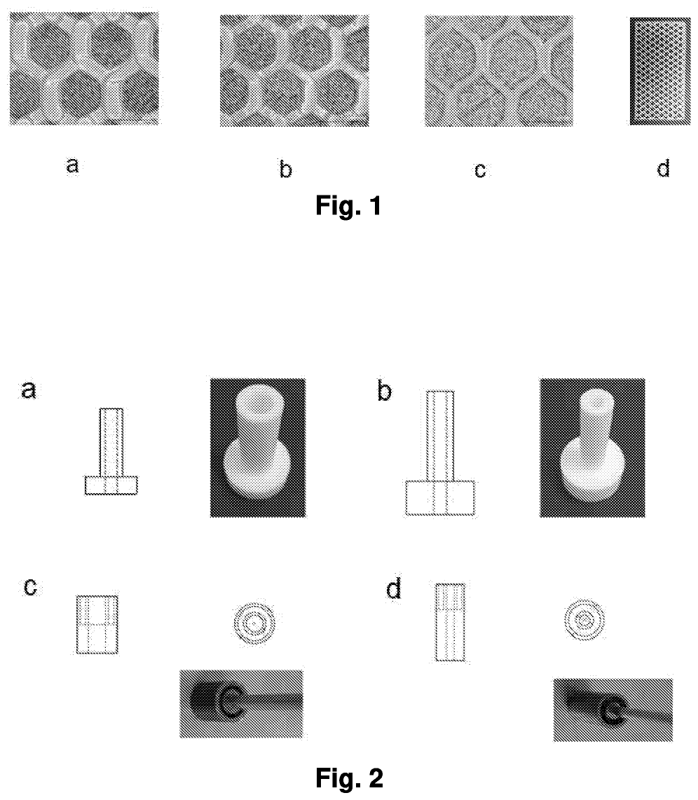

A Biopplotter 3D printer was used to print a PCL network mesh, first in a planar configuration, by heating and melting PCL into beads with a printhead suitable for high temperatures. Three different printhead speeds, namely 0.4, 0.6 and 1 mm/second, were used to obtain three different rectangular network meshes, with honeycomb-like pattern of different thread dimensions depending on the speed used. FIG. 1 a-c shows the enlarged images of a portion of the meshes thus obtained, while FIG. 1 d shows the image of the entire mesh obtained at a speed of 1 mm/s. The geometry of the mesh and the dimensions of the thread creating the honeycomb-like structure were chosen with a CAD program and converted to .stl before printing. Table 1 below reports the thread dimensions obtained for the three different printhead speeds:

TABLE 1

| Network mesh of PCL | Printhead | Honeycomb-like pattern |
|---|---|---|
| # | speed (mm/s) | thread dimensions (μm) |
| FIG. 1a | 0.4 | 620 ± 25.7 |
| FIG. 1b | 0.6 | 496 ± 22.6 |
| FIG. 1c | 1 | 296 ± 20.04 |

The rectangular planar network mesh obtained by 3D printing was subsequently rolled up to assume a cylindrical configuration, with the aid of water at a temperature of about 55° C. and subsequent cooling to room temperature to allow the network to maintain the new shape imposed, and be cut out with the necessary dimensions depending on the anatomy and size of the nerve to be coated with the guide channel for regeneration.

As for the support element, it was prepared as follows starting from commercially available chitosan from Heppe Medical GmbH (degree of deacetylation=92.6%, viscosity=151-350 mPas). The commercial product as such was used to prepare a 2% w/v aqueous solution with a 1% v/v acetic acid concentration. The solution was then stirred at 50° C. for 3 hours, then allowed to stir at room temperature overnight. The solution was then filtered through a nylon mesh, degassed overnight under vacuum, and stored at 4° C. until use.

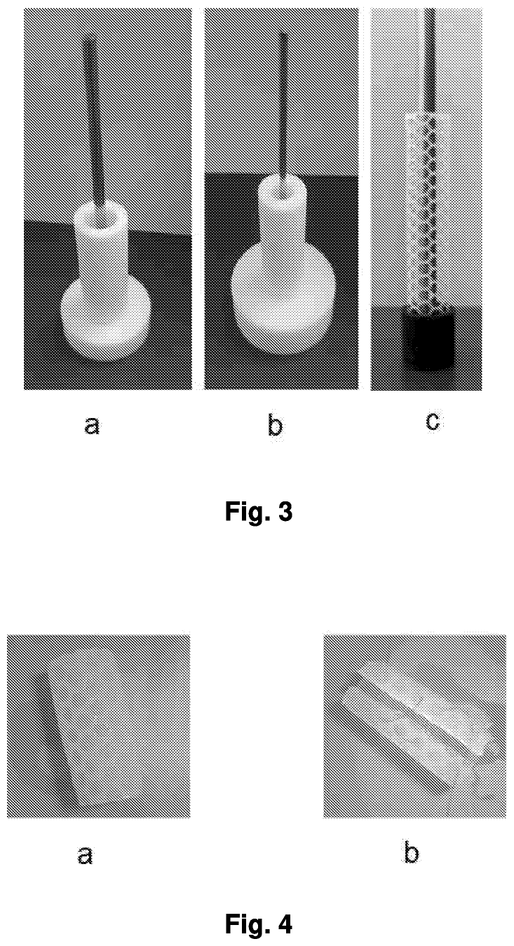

Using again a CAD program and the 3D printer mentioned above, molds of the desired shape, visible in FIG. 2, were then manufactured with two different geometries in terms of inner diameter and wall thickness to adapt to use in experiments on pigs (FIG. 2a,b) and on rats (FIG. c,d); each pair of molds was made of Teflon® (FIG. a,c) and of Delrin® (FIG. b,d). As can be seen in FIG. 2 b,d of these two molds were made with an internal groove adapted to accommodate the rolled PCL network mesh and in a vertical position. Steel rods measuring 3 mm and 1.5 mm in diameter for the larger and smaller molds respectively were then housed inside the molds to thus create the internal lumen in the guide channel for the experiments on pigs and for experiments on rats. FIG. 3 shows the Teflon® molds of different sizes with the steel rod (FIG. 3 a,b) and the PCL mesh inserted into the groove of a Delrin® mold. This network mesh was manually rolled up and placed inside a Teflon® cylinder, then incubated in water at 55° C. for 60 seconds before being inserted into the Delrin® mold, as shown in FIG. 3c. After this operation, the Delrin® molds were assembled to the respective Teflon® molds, leaving space for pouring the chitosan solution inside.

After filling the inside of the molds with the chitosan solution, the PCL mesh was perfectly immersed in the solution, and everything was transferred to the refrigerator allowing it to stand at a temperature of −20° C. or −80° C. for 12 hours until the solution solidified. The molds were then brought to room temperature for 10 minutes in order to thaw the external layer of water and then extract the steel rod and put in the freeze-dryer for 8 hours. After freeze-drying, a porous support was obtained and which was incubated first in 1% w/v sodium hydroxide for 15 minutes to neutralise the acetic acid and then in phosphate buffer saline (PBS) for 12 hours at room temperature to balance the pH of the support to the physiological value of the human body of 7.4. Once this step was also completed, the resulting guide channel was cut out according to the anatomical specifications required for the experiments. FIG. 4 a,b show photographs of two chitosan and PCL guide channels obtained with the above-described process, respectively in a tubular configuration whose cylinder is closed or open with a cuff-like longitudinal cut.

### Example 2

### Characterization of the Guide Channels of the Invention Prepared in Example 1

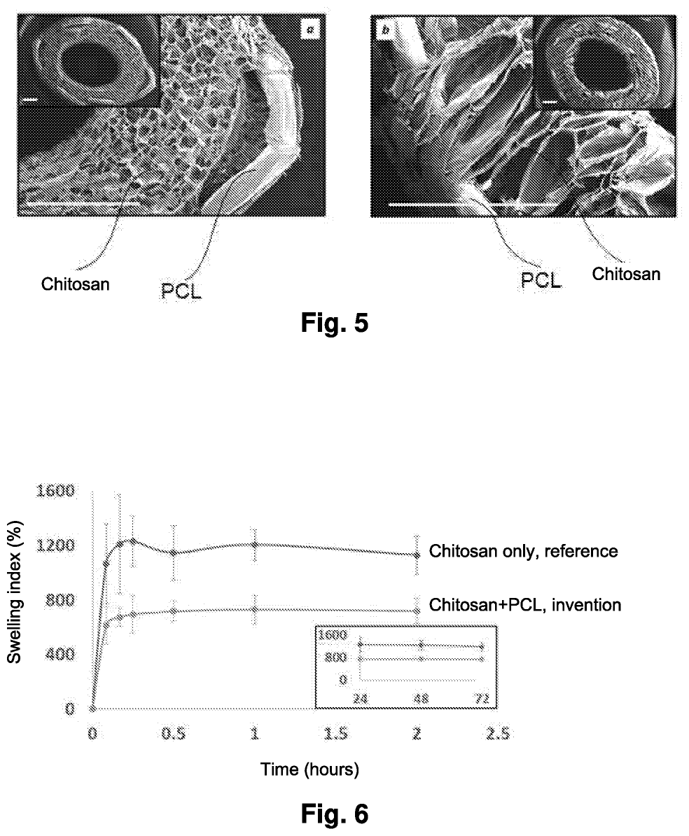

The chitosan and PCL guide channels prepared as described above in Example 1 were characterized to further study their structural and mechanical characteristics. FIG. 5 reports the images obtained by scanning electron microscopy (SEM) in which the microstructure of the guide channel, obtained at the two different freeze-drying temperatures tested of −20° C. and −80° C., is visible. In the images, the threads of the PCL network and the porous chitosan matrix are highlighted by arrows; the scale bars are 1 mm.

As can be appreciated from these SEM images, different microstructures of the polymer matrix are obtained depending on the freeze-drying temperature. The microstructure obtainable with the temperature of −20° C. shows randomly oriented polymer chains with isotropic pore structures (FIG. 5a), while the microstructure of the material obtained at −80° C. shows lamellar pores oriented in the radial direction with a more anisotropic shape (FIG. 5b). This change in the microstructure influences the porosity of the material, which was calculated to be 91.2±1.1% for the material obtained at −80° C., and 86.4±1.3% for the material obtained by setting the freeze-drying temperature at −20° C. These porosity values are characteristic of a structure with highly interpenetrated pores, which are essential for good adhesion of the cells and for their diffusion within the microstructure, therefore for obtaining advantageous conditions for the regeneration of the damaged nerve.

Table 2 below reports the dimensions of the guide channel obtained in the two different freeze-drying conditions for the experiments on pigs, in a dry condition and after swelling by incubation at 37° C. in PBS:

TABLE 2

| Freeze-drying | Dry | | Dry inner | Inner diameter | Swelling |
|---|---|---|---|---|---|
| temperature | thickness | Thickness after | diameter | after swelling | thickness |
| (° C.) | (mm) | swelling (mm) | (mm) | (mm) | (%) |
| −80 | 1.7 ± 0.14 | 1.87 ± 0.12 | 3.81 ± 0.3 | 3.48 ± 0.27 | 9.5 |
| −20 | 1.56 ± 0.24 | 1.89 ± 0.12 | 3.81 ± 0.44 | 3.47 ± 0.22 | 17.3 |

As indicated above in Table 2, as a result of hydration, the guide channel undergoes swelling with an increase in wall thickness by 9.5% for the material made at a freeze-drying temperature of −80° C. and by 17.3% for the material made at a freeze-drying temperature of −20° C. In contrast, the inner diameter by hydration decreased by about 9% in both cases.

This behaviour is characteristic of highly hydrophilic materials that absorb a large amount of water by immersion in a physiological medium. The kinetics of this hydration process was characterized and expressed in terms of percentage variation of the absorbed water content, or % swelling index, during the incubation time in PBS at 37° C. for a guide channel of the invention made with the PCL network mesh immersed in a porous chitosan matrix, prepared as described in Example 1, compared to the chitosan matrix alone for comparison. The results of this characterization for the two materials are reported in the graph in FIG. 6, where a rapid and enormous increase in volume of the chitosan matrix by water absorption is observed, with a swelling index of 1145%, while it is around 700% for the guide channel of the invention in which the PCL network mesh is immersed in the chitosan matrix, demonstrating that the PCL mesh stabilises the chitosan matrix avoiding excessive swelling. It is also interesting to note that for both materials the swelling indices reach equilibrium after about 30 minutes, without undergoing further variations and thus ensuring a constant shape and size over time, but the reference material, given the enormous swelling, does not have a suitable behaviour to support the electrode and promote nerve regeneration inside the human body.

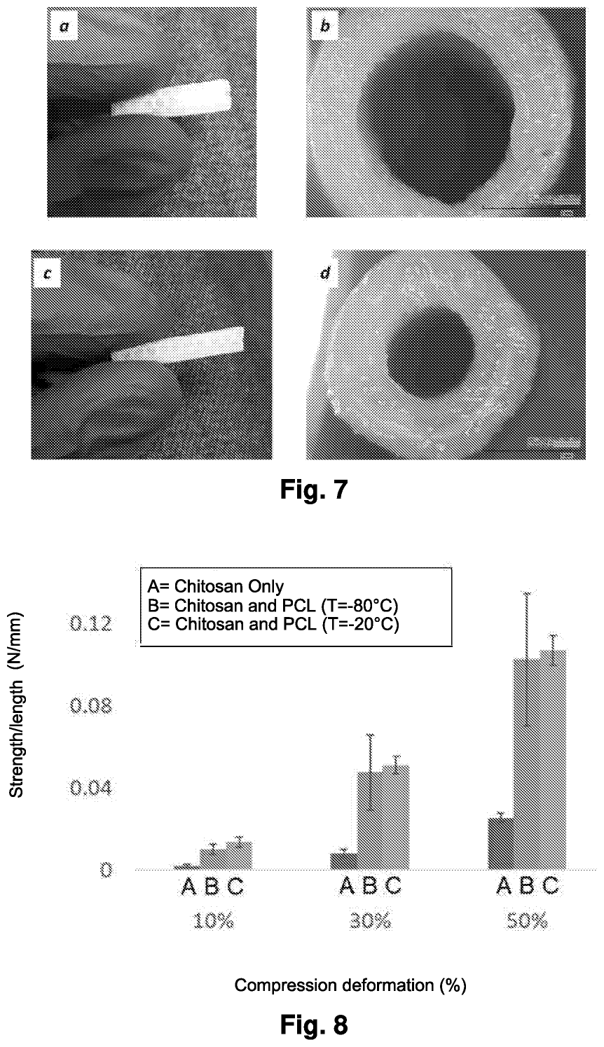

With the materials prepared as described above in Example 1 and swollen in PBS, various cutting tests were also carried out according to various designs, depicted in FIG. 7. FIG. 7 a,b show photographs of guide channels for experiments on pigs rescaled with thinner wall thickness, while FIG. 7 c,d show photographs of guide channels rescaled for experiments on rats. As can be seen in these photographs, by adapting the molds and the dimensions of the network mesh, it is possible to obtain different geometries and designs of the guide channel, in order to adapt the device of the invention to the anatomical variations of the nerves and to the selected animal model. For example, the guide channel for experiments in pigs (FIG. 7a,b) was designed to have a reduced wall thickness from 1.87+0.12 mm to 1.17+0.21 mm while for the experiments in rats (FIG. 7c,d) it was designed to have a reduced wall thickness from 1.05+0.08 mm to 1.95+0.17 mm.

A degradation characterization was also made to verify that the guide channel of this invention can remain stable under physiological conditions at least for a period of time comparable to the time necessary to complete the nerve regeneration process, which has been observed to occur several weeks after implantation of the device in a variable time depending on the animal model and nerve injury. For this purpose, the inventors performed experiments by incubating the guide channel as prepared in Example 1 in PBS at 37° C. with a 4 mg/ml lysozyme concentration. The enzyme lysozyme can degrade chitosan in a physiological environment and the degradation rate thereof is inversely proportional to the degree of deacetylation of the chitosan molecule. On the other hand, the guide channel of this invention remained stable after 1 month of incubation, resulting in a weight loss of about 10% by weight compared to the initial weight, thus confirming that the chitosan matrix remains stable for the period necessary for the nerve regeneration process, which is of the same order of magnitude for an interruption of nerve continuity of about 10 mm, based on the characterizations carried out in vivo on rats.

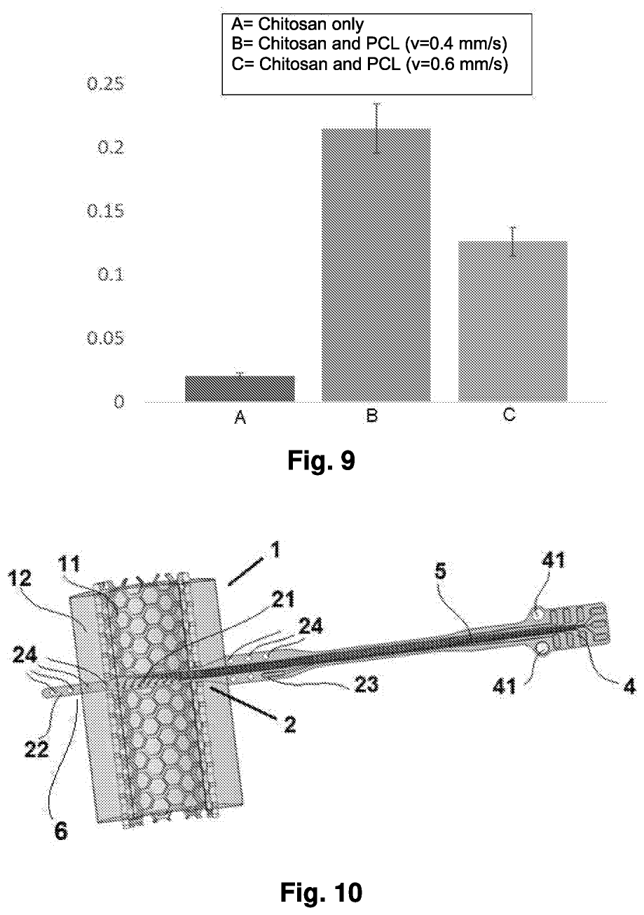

Furthermore, in order to confirm the mechanical properties of the guide channel of the invention, the inventors investigated the contribution of the PCL network mesh to the improvement of the resistance of the guide channel during the implantation period of the device. The compression resistance and the bending stiffness of different guide channels were evaluated in the animal model of a pig implanted with the devices. The results of this evaluation are illustrated in FIGS. 8 and 9, where the compression resistance and the results of the 3-point bending test for the chitosan matrix alone without PCL network mesh, and for chitosan with PCL network prepared at different freeze-drying temperatures and printing speed of the PCL network, respectively, are reported. As can be noted in FIG. 8, the PCL network mesh is the predominant component in the mechanical behaviour of the guide channel made with chitosan matrix and this network immersed in the matrix. This confirms the validity of the choice of combining the matrix with the network mesh not only to improve the mechanical performance of the guide channel, but also to give the appropriate stability to the thin film electrode placed transversely within the lumen of the channel. In particular, FIG. 8 shows how the PCL network mesh increases the compression resistance of the guide channel, especially at compression resistance values of 30% and 50%, where the mesh is able to increase the resistance by 587% and 408% respectively for the guide channel made at a freeze-drying temperature of −80° C.

With regard to the results of the 3-point bending test reported in FIG. 9, it can be noted that not only does the PCL network mesh consistently increase the bending stiffness, but this value depends on the width of the PCL thread of the mesh. The average bending stiffness of the guide channel with the thinnest PCL thread is 0.126 N mm, while this value increases up to an average of 0.215 N mm for the thicker PCL threads, incorporated into the porous chitosan matrix. Therefore, by varying the thickness of the PCL threads in the network mesh by simply changing the printing patterns, it will be possible to modulate the bending stiffness of the guide channel and adapt it to the anatomical characteristics of the body district in which the device is to be implanted, including the anatomical areas that are more complicated to manage because they are very narrow, such as the thoracic portion of the vagus nerve where body movements and weight can cause the guide channel to collapse, resulting in electrode rupture and failure of nerve regeneration. A guide channel of the invention, the stiffness of which can be easily modulated when printing the PCL network mesh, will also be able to adapt to these districts, minimising the risks of electrode rupture and medical treatment failure.

### Example 3

### Construction of a Thin Film Electrode for the Regenerative Nerve Interface Device of the Invention

The thin film electrode used for the experiments with the guide channel prepared as above was a particular embodiment of the present electrode, double-sided, wherein each side had 9 active contacts and 1 ground (GND hereinafter). The two sides, mirroring each other, were glued together to obtain a double-sided electrode with a total of 2 GNDs and 18 active contacts, circular in shape and 80 μm in diameter, arranged so as to be inside the lumen of the guide channel, once the device is implanted, to exert stimulation on the nerve. The set of active contacts was placed transversely so as to intercept the different fascicles being regenerated in the entire diameter of the guide channel where the nerve is passed. The GND electrodes are located outside the guide channel in the device to be implanted along the conductive wires that connect the active contacts on the thin film to a printed circuit board (PCB).

FIG. 10 schematically illustrates a guide channel 1, with the PCL network mesh 11 immersed in the chitosan tubular matrix 12, and a thin film electrode 2 in which the active contacts 21, a distal end 22 and a proximal end 23 both provided with holes 24, which can be used to secure the device with suture thread to the tissues surrounding the anatomical district where the device is implanted, can be distinguished. In FIG. 10, the electrode 2 is connected by conductive wires 3 to a printed circuit board 4 as a control unit, which is also provided with holes 41 to secure it to the tissues with suture thread. GND ground electrodes are indicated by 5, while 6 indicates the point where the electrode is glued to the guide channel with biocompatible, hardenable glue, as described in more detail below.

The polyimide structure with the active contacts was prepared in the clean room using photolithographic procedures, comprising steps 1 to 15 as illustrated in detail in FIG. 11. Briefly, a silicon wafer (Si-Mat) was used as a substrate for the entire layer-on-layer lithographic process. Polyimide PI2610 (HDMicroSystem) was spun twice at 2200 rpm for 30 seconds to obtain a final layer of 6 μm thickness (approximately 2.9 μm for each layer), which was then oven-hardened for 3 hours at 360° C. under nitrogen atmosphere (FIG. 10-1). The lift-off layer and the photoresist layer (LOL2000 and S1813, Microposit) were deposited by spinning on the wafer at 1000 rpm for 20 seconds and 4500 rpm for 30 seconds, respectively (FIG. 10-2.3), and then they were exposed to UV radiation (189 mJ cm⁻²) (Karl Suss) using a glass photomask (FIG. 10-4). The samples were then developed using MF-319 (MicroChem) for 30 seconds, and rinsed with deionised water (FIG. 10-5). Oxygen plasma (30 seconds, 150 W, 15 sccm O₂, 300 mTorr) was used to functionalise the surface and improve the adhesion of additional metal layers deposited by sputtering. A 25 nm-thick titanium nanolayer (150 W, 1.5 min) and a 250 nm-thick gold layer (100 W, 2 min) were then deposited by sputtering in a 4×10⁻⁶ Torr vacuum (FIG. 10-6), and then a lift-off was made by dipping the wafer into the photoresist layer removing agent (FIG. 10-7). The samples were again washed with deionised water and dried. An additional polyimide layer (2.9 μm thick) was deposited by spinning and hardened at high temperature (FIG. 10-8) as described above. An aluminium layer (200 nm) was then thermally evaporated on the wafer (1×10⁻⁶ Torr; 1.3 A) (FIG. 10-9).

A photoresist layer (S1813, Microposit) was deposited by spinning (FIG. 10-10), exposed to UV radiation (FIG. 10-11) and developed as mentioned above (FIG. 10-12). The aluminium layer was wet etched (FIG. 10-13) with a solution of HNO₃:H₃PO₄:HAc:H₂O 4:1:1:4 for 30 minutes at room temperature. The samples were finally subjected to dry etching using oxygen plasma with an oxygen flow of 30 sccm at 150 W for 65-75 minutes (FIG. 10-14), and the aluminium mask was removed using the solution described above (FIG. 10-15). The samples were finally washed with deionised water and dried, then the films were gently removed from the wafer with tweezers, and used to construct the electrode as described above and schematically illustrated in FIG. 10.

### Example 4

### Characterization and Validation of a Thin Film Electrode for the Regenerative Nerve Interface Device of the Invention

The thin film electrode constructed as described above in Example 3 was subjected to characterization and validation using electrochemical impedance spectroscopy (EIS), cyclic voltammetry (CV), and voltage transient measurements (VTm), as described below.

80 ml of PBS was kept under nitrogen for 20 minutes to eliminate oxygen. Then three thin film electrodes prepared as described above were immersed in this solution so that only the active contacts were immersed. A sinusoidal alternating current of 5 mV RMS was then passed to measure the impedance in a range from 10 Hz to 100 kHz, at a physiologically reasonable frequency of 1 kHz. The CV was done to evaluate the cathodic charge storage capacity (CSC) of the active contacts of the electrodes constructed as described above, using Matlab program functions (MathWorks). The cycles were conducted with a scan rate of 1 V/s, between-0.6 and 1 V, as potential limits; remaining within this range was in fact important to prevent oxidation or water reduction processes. Voltage transient measurements were made with rectangular, biphasic current pulses of an amplitude set at 500 us, like in the in vivo stimulation parameters. The current amplitude ranged from 10 to 500 μA. The resulting VTm data were analysed in Matlab to obtain the highest possible current at which the reduction limit of-0.6 V was not exceeded by the electrode potential. The calculation of the maximum injectable charge (mx Q-inj) was also done using Matlab, as precedingly described by Cisnal et al. (2018), where the maximum injectable charge is defined as the maximum charge that can be injected into a current-controlled stimulation pulse, without damaging the material of the active contacts or the coating.

The electrochemical analysis showed that, at 1 KHz, the active contacts of the electrodes prepared as described above have an average impedance of 2.4-5.8 k (2 (std+/−2.5). The charge storage capacity was 448.3 mC/cm². The maximum Q-inj was 6.9 nC (std+/−14.4) per active contact. The characterization of the structure of the polyamide thin film resulted in a thickness of about 7.5 μm, as expected, while the actual measured height was 6.4 μm. In conclusion, the electrochemical characterization showed that the electrode prepared as described above was functional and ready to be integrated into the guide channel.

### Example 5

### Preparation of the Regenerative Nerve Interface Device of the Invention

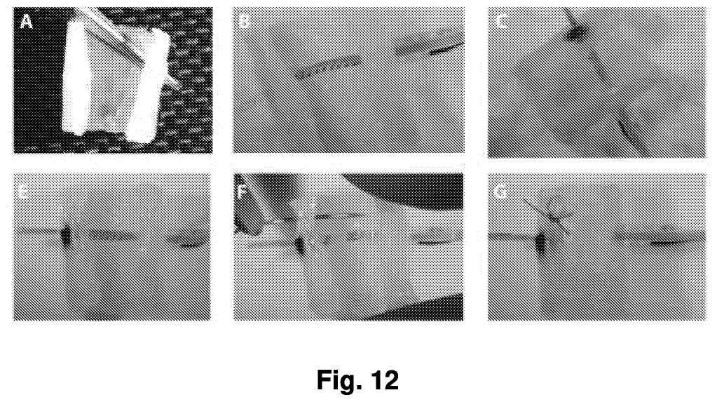

The electrode constructed as described above in Example 3 and the guide channel prepared as described in Example 1 were assembled by the following procedure, illustrated herein with reference to FIG. 12. A guide channel, in a cuff-like configuration, was drilled with tweezers, creating two holes on opposite sides on the cylindrical surface of the channel (FIG. 12-A), in order to be able to insert the electrode so that the active contacts of the electrode are placed in the centre of the channel (FIG. 12-B). Then, once the electrode was placed in position, biocompatible glue 1401-M-UR (Dymax) was applied to fix the distal end of the electrode to the guide channel (FIG. 12-C). By applying a source of UV radiations for about 60 seconds, the glue was hardened (FIG. 12-E). Finally, the guide channel was closed to create a closed tubular structure by joining the two open ends with suture thread (FIG. 12-F,G).

The present invention has been described herein with reference to a preferred embodiment. It is to be understood that there may be other embodiments that relate to the same inventive core, within the scope of protection of the claims provided below.

### BIBLIOGRAPHICAL REFERENCES

[1] J. del Valle, X. Navarro, Interfaces with the peripheral nerve for the control of neuroprostheses, Int. Rev. Neurobiol. 109 (2013) 63-83.
[2] X. Navarro, T. B. Krueger, N. Lago, S. Micera, T. Stieglitz, P. Dario, A critical review of interfaces with the peripheral nervous system for the control of neuroprostheses and hybrid bionic systems, J. Peripher. Nerv. Syst. 10 (2005) 229-258.
[3] N. Lago, D. Ceballos, F. J Rodriguez, T. Stieglitz, X. Navarro, Long term assessment of axonal regeneration through polyimide regenerative electrodes to interface the peripheral nerve, Biomaterials. 26 (2005) 2021-2031.
[4] A. E. Rochford, A. Carnicer-Lombarte, V. F. Curto, G. G. Malliaras, D. G. Barone, When Bio Meets Technology: Biohybrid Nerve interfaces, Adv. Mater. 1903182 (2019) 1903182.
[5] B. S. Spearman, V. H. Desai, S. Mobini, M. D. McDermott, J. B. Graham, K. J. Otto, J. W. Judy, C. E. Schmidt, Tissue-Engineered Peripheral Nerve Interfaces, Adv. Funct. Mater. 28 (2018) 1-18.
[6] S. P. Lacour, S. Benmerah, E. Tarte, J. Fitzgerald, J. Serra, S. McMahon, J. Fawcett, O. Graudejus, Z. Yu, B. Morrison, Flexible and stretchable micro-electrodes for in vitro and in vivo nerve interfaces, Med. Biol. Eng. Comput. 48 (2010) 945-954.
[7] I. Delgado-Martínez, M. Righi, D. Santos, A. Cutrone, S. Bossi, S. D'Amico, J. Del Valle, S. Micera, X. Navarro, Fascicular nerve stimulation and recording using a novel double-aisle regenerative electrode, J. Neural Eng. 14 (2017).
[8] Larson, C. E., & Meng, E. A review for the peripheral nerve interface designer. Journal of neuroscience methods, 332, (2020). 108523.
[9] US20180338765A1.

1. A tubular guide channel for supporting a nerve to be regenerated in regenerative nerve interface devices, said guide channel comprising a tubular element comprising a hydrophilic natural polymer, biocompatible and biodegradable, in form of a porous matrix, and a mesh comprising a synthetic thermoplastic polymer, biocompatible and biodegradable, immersed in said tubular element, and an internal lumen of size suitable for housing said nerve to be regenerated.

2. The tubular guide channel of claim 1, wherein said network mesh extends within said tubular element over the entire surface.

3. The tubular guide channel of claim 1, wherein said natural polymer is selected from the group consisting of chitosan, collagen, hyaluronic acid, sodium alginate, dextran, cellulose, pectin, agarose, gellan, xanthan gum, silk, fibrin, keratin, chondroitin sulfate, and mixtures thereof.

4. The tubular guide channel of claim 1, wherein said natural polymer is chitosan.

5. The tubular guide channel of claim 1, wherein said synthetic polymer is selected from poly(ε-caprolactone)(PCL), polylactic acid (PLA), polyglycolic acid (PGA), copolymers of polylactic acid and polyglycolic acid (PLGA), and mixtures thereof.

6. The tubular guide channel of claim 1, wherein said synthetic polymer is PCL.

7. The tubular guide channel of claim 1, wherein said channel has a closed cylinder configuration or a cuff-like, longitudinally open cylinder configuration.

8. The tubular guide channel of claim 1, wherein said tubular element and/or said mesh further comprises one or more agents selected from anti-inflammatory agents, antibiotics, antioxidants, vitamins, agents assisting nerve regeneration.

9. The tubular guide channel of claim 8, wherein said agents are incorporated in said tubular element and/or in said mesh in the form of micro- or nano-particles formulated as a powder, solution, or dispersion, optionally as a time-controlled release of a pharmaceutical formulation.

10. A regenerative nerve interface device comprising a tubular guide channel of claim 1 and a thin film electrode having an array of electrically active contacts, a distal end and a proximal end, said electrode being transversely inserted inside said guide channel so that said distal and said proximal ends emerge from opposite surfaces of said guide channel outwards, while said array of active contacts is placed within said guide channel.

11. The device of claim 10, wherein said thin film electrode comprises a printed circuit board coated with a polyimide film in which one or more conductive tracks of one or more conductive metal layers are engraved.

12. The device of claim 11, wherein said one or more conductive metal layers are made of gold.

13. The device of claim 10, wherein said one or more conductive metal layers are further coated with one or more conductive layers selected from iridium oxide and conductive polymers.

14. The device of claim 10, wherein said distal end and/or said proximal end comprise one or more holes adapted to anchor said device to surrounding tissues by means of suture thread.

15. A system for regenerating a nerve comprising the regenerative nerve interface device of claim 10, means for controlling and processing electrical signals coming from said device, means for the electrical connection between said device and said control and processing means.

16. The composition of claim 1, wherein the system of claim 15, wherein said means for controlling and processing electrical signals comprises a printed circuit board.

17. The system of claim 15, wherein said electrical connection means are conductive wires.

18. The system of claim 15, further comprising a ground electrode placed between said thin film electrode and said control means.

19. The system of claim 15, wherein at least said control and connection means are inserted within a sheath of biocompatible material, optionally silicone, comprising one or more holes adapted to anchor said system to surrounding tissues by means of suture thread.

20. A process for preparing a regenerative nerve interface device of claim 10, comprising the steps of:
i) providing a network mesh of thermoplastic synthetic polymer by 3D printing at a predetermined printhead speed; and an aqueous solution of natural hydrophilic polymer;
ii) providing a mold adapted to form a cylindrical matrix of said natural polymer with an internal lumen, optionally provided with a groove to hold said network mesh in place;
iii) inserting said rolled network mesh into said mold and pouring said solution of natural polymer;
iv) subjecting the mold to freeze-drying at a predetermined temperature, and extracting the guide channel comprising a tubular element comprising said natural polymer in the form of a porous matrix and a network mesh comprising said synthetic polymer immersed in the matrix;
v) optionally bringing the guide channel to about 7.4 physiological pH by incubation in a suitable aqueous solution;
vi) providing a thin film electrode having an array of electrically active contacts, a distal end and a proximal end; and
vii) inserting said electrode transversely within said guide channel so that said distal and said proximal ends emerge from opposite curved surfaces of said guide channel outwards while said array of electrically active contacts is placed within said guide channel.

21. The process of claim 20, further comprising a step of fixing said thin film electrode to said guide channel by gluing with a biocompatible glue, optionally followed by a step of hardening the glue, or by suturing said electrode to said network mesh.

22. The process of claim 21, wherein said gluing is carried out at the point of said guide channel from which said distal end of the electrode emerges outwards of the channel.

23. The process of claim 1, wherein said freeze-drying temperature is between −20° C. and −200° C.

24. The process of claim 1, wherein varying said printhead speed modulates the weave of the network mesh, the thickness of the thread that composes it and consequently the bending stiffness of the guide channel.
